(19) United States
(12) Patent Application Publication
(10) Pub. No.: US 2009/0030489 A1
(43) Pub. Date: Jan. 29, 2009

(54) BODY COVER, GLASSES AND/OR AT LEAST PARTIAL HEAD COVER, METHOD FOR RADIATING AT LEAST PART OF A HUMAN BODY AND USE OF A BODY COVER

(75) Inventors: Sima Asvadi, Eindhoven (NL); Bernardus Hendrikus Hendriks, Eindhoven (NL); Philomeen Antoinette Catharina Engels, Delft (NL); Gerrit Jan Van Den Beld, Eindhoven (NL); Jan De Graaf, Eindhoven (NL); Henk Van Houten, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/278,286

(22) PCT Filed: Jan. 30, 2007
(86) PCT No.: PCT/IB07/50305

§ 371 Date: Aug. 5, 2008

(30) Foreign Application Priority Data
Feb. 6, 2006 (EP) 0603887.4

(51) Int. Cl. A61N 5/06

(52) U.S. Cl. 607/88

(57) ABSTRACT

Body cover, comprising a substrate, for covering at least part of a human body, and at least one electroluminescent source coupled to the substrate, for illuminating at least a part of the body, wherein the cover is configured to at least partly surround at least a part of the body and/or a body support. Method for radiating at least part of a human body, especially in the treatment of jaundice and/or crigler najjar, comprising covering at least part of the body and/or a body support with a cover with at least one electroluminescent source and emitting light from the electroluminescent source to at least part of the body.

### FIELD OF THE INVENTION

The invention relates in general to a body cover, comprising a substrate, for covering at least part of a human body, and at least one electroluminescent source coupled to the substrate.

### BACKGROUND OF THE INVENTION

Light is known to have beneficial effects on humans. In fields such as phototherapy light can be used for healing purposes. A case where light treatment is used is hyperbilirubinemia. Hyperbilirubinemia patients are daily exposed to a high dose of blue light for a period up to eleven hours. Hyperbilirubinemia is a disorder of bilirubin metabolism. Bilirubin is the toxic waste product of the worn-out red blood cells that get converted by an enzyme produced by the liver, which excretes the bilirubin. Hyperbilirubinemia patients lack this enzyme, which causes an accumulation of the bilirubin in the tissue. This results in jaundice of the skin and the eyes. Neonatal jaundice and crigler najjar are two types of Hyperbilirubinemia. Crigler najjar patients range from the age of infant to adult and need daily light therapy for life, for various periods as long as 11 hours a day. Neonatal patients oftentimes need continuous light therapy for a few days.

Bilirubin degrades under influence of phototherapy. The therapeutic efficacy of phototherapy is, among others, dependent on the spectral light qualities, the intensity of the light, the exposed body surface area and the duration of exposure. A high-intensity narrow band blue light is used with a wavelength in the 450-470 nm spectrum, centered around a 450 nm peak and no light emission in the UV wavelength area. Light intensities of at least 5 mW/cm² up to 10 mW/cm² and full body exposure are used for keeping treatment durations relatively acceptible.

Known treatments for jaundice, particularly baby jaundice, combine a warming box with light treatment. The warming box is required since the baby has to be naked for the efficiency of the light treatment, making the normal process of taking care of the baby, for example breast feeding, more difficult and limiting its freedom of movement. The eyes of the baby are covered to be protected against bright light.

Known phototherapy devices for treatment of hyperbilirubinemia commonly use fluorescent tubes or halogen spots. Disadvantages of these devices include physical size and shape, heat production, unstable and broad wavelength light output, limited body surface area illumination and UV radiation. The conventional phototherapy devices illuminate the patients from one side, for example, because it is uncomfortable to lie on a transparent plastic sheet or transparent fabric over the hot lamps for a long period. In some cases a transparent bed provides the opportunity for exposing the body from both sides, however, this bed doesn't meet the requirements of a comfortable bed.

Furthermore, a small fibreoptic sheet to illuminate a baby is developed. Drawbacks are the limited size and relative low intensity. Hence it is commonly used in combination with other phototherapy devices and besides, it can not be used for older patients.

Furthermore it is said that light having a wavelength of approximately 450 nm emitted to human eyes has a revitalising effect on a person. It is also known that phototherapy is used for stimulating purposes of cells in/near the skin, for example in wound healing, at different wavelengths within a wide range up to the infrared region.

### SUMMARY OF THE INVENTION

An object of the invention is to provide a relatively comfortable way of applying phototherapy.

This object and other objects are achieved by a body cover, comprising a substrate, for covering at least part of a human body, and at least one electroluminescent source coupled to the substrate, for illuminating at least a part of the body, wherein the cover is configured to at least partly surround at least a part of the body and/or a body support.

Said object and other objects are also achieved by glasses and/or at least partial head cover, connected and/or provided with a power supply for supplying power to the at least one electroluminescent source, wherein said glasses and/or said at least partial head cover are configured to have the at least one electroluminescent source emit light approximately into the eyes, preferably approximately near the bottom eye lids, wherein the at least one electroluminescent source has a wavelength between 440 nm and 480 nm, preferably between 447 nm and 470 nm, more preferably approximately 450 nm.

Furthermore, said object and other objects are also achieved by a method for radiating at least part of a human body, especially in the treatment of jaundice and/or crigler najjar, comprising covering at least part of the body and/or a body support with a cover with at least one electroluminescent source and emitting light from the electroluminescent source to at least part of the body.

### BRIEF DESCRIPTION OF THE DRAWINGS

FIG. 1 shows a diagram of a cover;

FIG. 2 schematically shows a top view foil with LEDs;

FIG. 3 schematically shows a perspective view of an interwoven yarn structure;

FIGS. 4, 5 and 6 show a cover;

FIGS. 7 and 8 show a cover as a sleeping bag;

FIG. 9 shows a cover as a sleeping bag for babies;

FIG. 10 shows a cover as a sleeping bag for infants;

FIG. 11 shows a modular cover for different sizes;

FIG. 12 shows a cover as a suit;

FIG. 13 shows a cover as a suit with a hood;

FIG. 14 shows a cover with an air cooling system;

FIG. 15 shows a detail of the air cooling system;

FIG. 16 shows a cover with a liquid cooling system;

FIG. 17 shows a mattress with a cover;

FIG. 18 shows a cover with arcs;

FIG. 19 shows a cover as a visor with LEDs;

FIG. 20 shows a cover as glasses with LEDs;

FIG. 21 shows a cover as a bra with LEDs;

FIG. 22 shows a cover as a lining for a bra with LEDs;

FIG. 23 shows a cover as a bra with LEDs.

In this description, identical or corresponding parts have identical or corresponding reference numerals. The exemplary embodiments shown should not be construed to be limitative in any manner and serve merely as illustration.

### DETAILED DESCRIPTION OF THE EMBODIMENTS

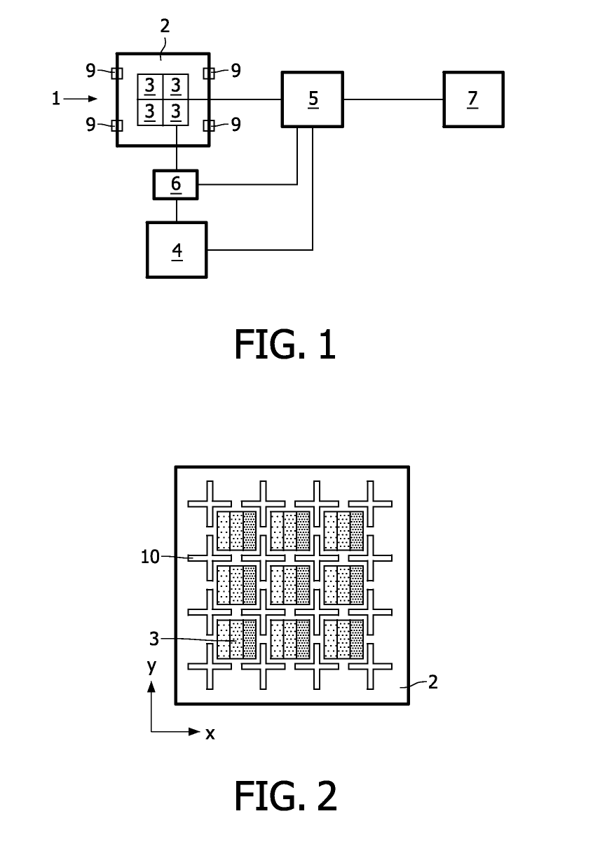

An embodiment of a cover 1 with electroluminescent sources for illuminating at least part of the body is shown in FIG. 1. The cover 1 comprises a substrate 2 which has electroluminescent sources 3 attached. The electroluminescent sources 3 are individually driven by power supply 4, which is controlled by control means 5. To individually address the electroluminescent sources 3, the power supply 4 may comprise a driver unit. Wavelength adjusting means 6 are provided for the possibility of varying the wavelength of at least a part of the electroluminescent sources 3 and are controlled by control means 5. By means of a user interface 7 the control means 5 can be set and/or adjusted to specify the settings of the electroluminescent sources 3. For instance by signalling the wavelength adjusting means 6, the wavelength of the electroluminescent sources 3 can be individually controlled, and/or it can be controlled in groups and/or all the sources 3 can be controlled. Also, by signalling the driver unit, the light output of the sources 3 can be controlled and/or turned on and off, for example.

The wavelength can for example be varied by turning on and off different electroluminescent sources 3 with different wavelengths. It is also possible to apply wavelength filters. By selecting the proper filters the proper wavelength can be selected. It is also possible to change the wavelength by varying the temperature of LEDs 3. Other wavelength adjusting means include using phosphors, preferably in combination with PWM (pulse width modulation) or varying the forward current, also preferably in combination with PWM. PWM can for example be applied to make sure that the intensity stays as desired while the wavelength is adjusted. Furthermore, the intensity of the light of the electroluminescent sources 3 can for example be set by means of adjusting the current through the power supply 4 for varying the light output. Of course, there are more ways of varying intensity, such as, by using PWM and/or by turning an amount of the electroluminescent sources 3 on/off, etc. The electroluminescent sources 3 are configured to emit therapeutic light to a part of a human body. Therefore, the cover 1 may for example be wrapped around a part of a human body, and/or around a body support such as a pillow, bed, chair, etc. In an advantageous embodiment the cover 1 is flexible and may be folded so that it is portable. Joining means 9 that join together are provided in an advantageous embodiment of the cover, so that the cover 1 can be configured in a bag or cylinder shape and the part of the body can be surrounded by light from the electroluminescent sources 3. Joining means 9 may for example comprise, buttons, zippers, velcro, elements that can be tied together, elastic means for closing around at least a part of a body and/or body support, etc.

In an embodiment of the cover 1, the electroluminescent sources 3 comprise light emitting sources like LEDs (Light Emitting Diodes) 3, for example gallium-nitride blue LEDs. The latter are relatively effective, safe and convenient phototherapy devices and capable of delivering high intensity narrow wavelength band light. Furthermore, they can be configured into flexible devices, such as cover 1. In an embodiment, the cover 1 is provided with a substrate 2 that is made of a flexible foil 2 with LEDs 3 attached to it. Examples of such a flexible foil 2 provided with LEDs 3 are described in non-prepublished European patent application EP 0510470.3. An embodiment of the foil 2, at least, a part of a foil 2, with LEDs 3, is shown in FIG. 2. As can be seen from FIG. 2 the foil 2 is provided with rows and columns of openings 10, between the LEDs 3. The foil 2 can be constructed in different sizes. The openings 10 of the foil 2 increase flexibility and the breathing properties of the cover 1. Different embodiments of the foil 2 for example comprise knitted, woven and non-woven textiles and/or polymers, rubbery materials, spacer fabrics, foams, etc. The foil 2 is provided with not shown conducting lines of an electrical circuit, between the openings 10, for supplying power to and/or controlling the LEDs 3.

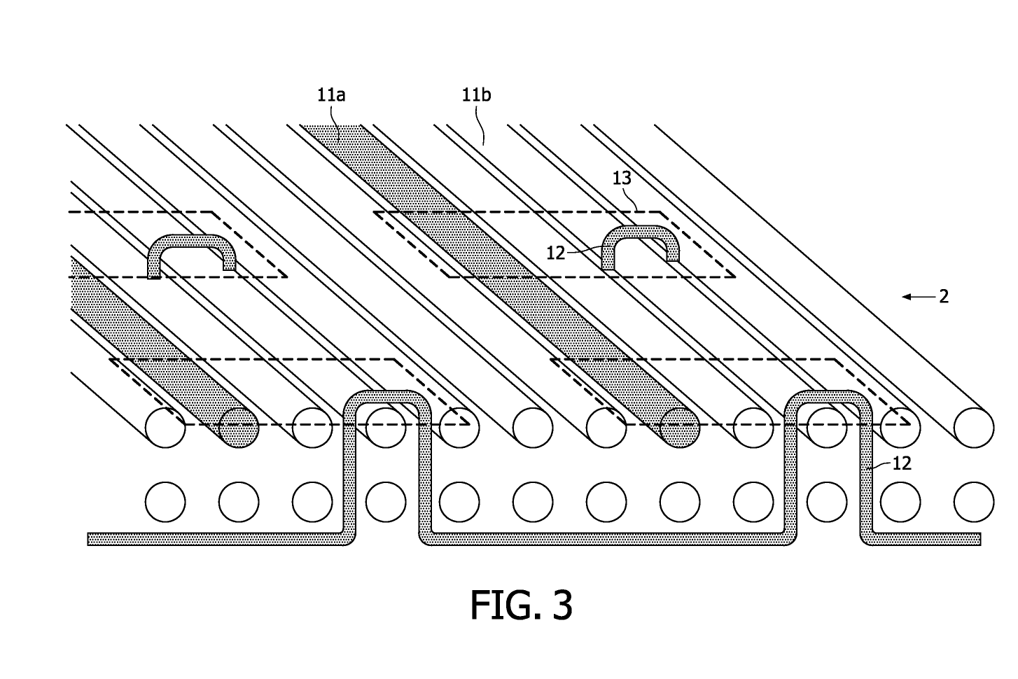

Another embodiment of the cover 1 is provided with LEDs 3 that are attached to yarns 11, 12 in a woven structure 2 of conducting and non-conducting yarns 11, 12. Such an embodiment is for example described in non-prepublished European patent application EP 05104703.3. FIG. 3 shows such an embodiment wherein the substrate 2 comprises a woven structure 2 of yarns 11, 12. The woven structure 2 is provided with conducting yarns 11a, 12 which comprise anodes 11a and cathodes 12. LEDs (not shown in FIG. 3) are connected with these conducting yarns 11, 12 at locations 13. In an embodiment, the configuration of the structure 2, more particularly, the yarns 11, 12, is such that the LEDs can be individually addressed. The structure 2 can comfortably cover a part of the body and illuminate it at the same time. The structure 2 can for example be a textile and can be combined with other (transparent) layers of textile, foil, polymers, etc. for example for extra protection of the body. Of course, the woven structure 2 doesn't have to be a textile but may also comprise a polymer such as nylon and/or polyester, etc. The structure 2 of this embodiment is flexible and can have the properties of body covers like clothes.

In another embodiment the electroluminescent sources 3 comprises OLEDs (organic LEDs) 3. OLEDs 3 are electroluminescent sources that can be configured to be flexible and/or transparent. Also, one OLED 3 can cover a large area of the body without being interrupted, while maintaining flexibility, so that a diffusing layer is not necessary, as opposed to many LEDs. The OLED 3 covering said large area can be driven by just one set of electrodes. The cover 1 can be provided with one or multiple OLEDs 3. OLEDs 3 are known in the art, and are for example described in the non-prepublished European application EP 05110643.3.

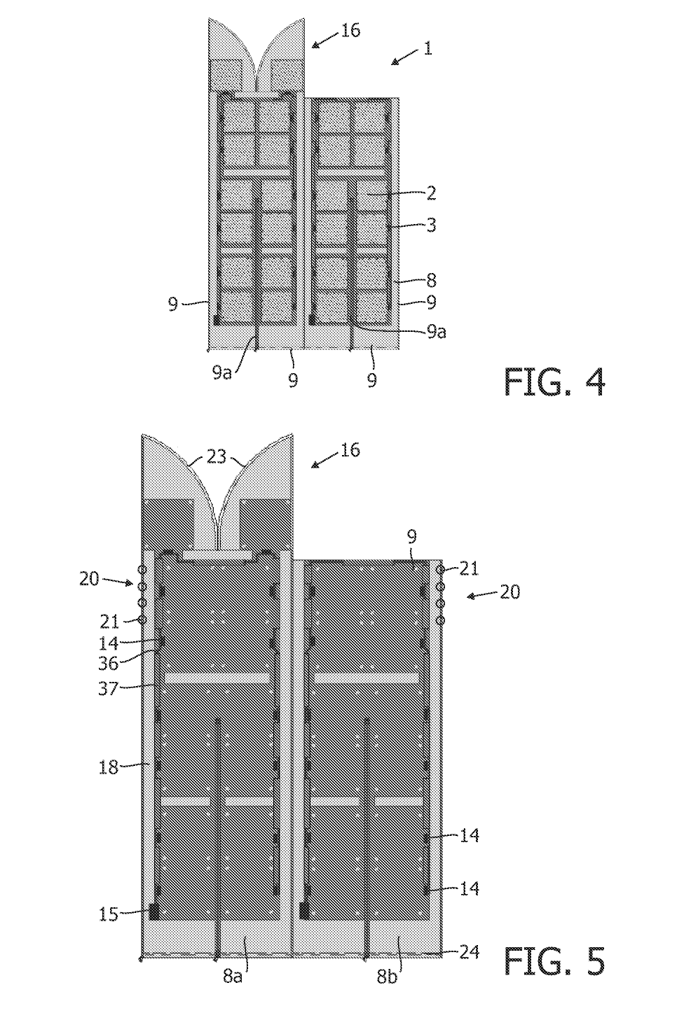

An embodiment of the cover 1 is shown in FIG. 4. This embodiment comprises a flexible sheet 8 with foils 2 attached on the inside, for example, but not limited to foils 2 such as the foil 2 with LEDs 3 that is shown in FIG. 2. LEDs 3 (not shown in FIG. 2) are attached to the foil 2. A zipper 9 is provided along the edges of the sheet 8, so that the sheet 8 can be formed into a bag, for example a kind of sleeping bag. For example, the sheet 8 is made of a breathable material and contains strips of net material to enhance ventilation. In an embodiment, the edges 18 of the sheet 8 are made of a very breathable gauze material. Also pants forming zippers 9a may be provided, adding a hybrid function to the cover. FIG. 5 shows such a cover 1, where the foils 2 with LEDs 3 are taken off. The foils 2 are attached to the sheet 8 by means of attachment means such as buttons 9, or for example velcro, magnetic means, static electricity, sticking means, etc. so that they are exchangeable, for example for washing or changing a defect foil 2 or for washing the cover 1 without damaging the LEDs 3 and/or the electrical circuit of the foil 2. When attached, the foils 2, more particularly, the LEDs 3, connect with the foil connectors 14 that are provided at the location of where the foils 2 will be attached, at least approximately. The connectors 14 can also be integrated with said attachment means 9. The foil connectors 14 are connected to a plug contact 15, through which a connection is made with power supply 4 and in a particular embodiment, with control means 5. The foil connectors 14 are for example connected to the contact 15 by means of conducting lines or a cable 36. The cable 36 may be kept in place by straps 37 in the cover 1 and can easily be removed from the cover 1. In this way, the individual foils 2, more particularly the LEDs 3, can be driven and controlled by making simple contact with the foil connectors 14. For illumination around, or on the head or shoulders, flaps 16 are fixed to the cover 1.

In different embodiments, the power supply 4 comprises a battery 4, for example, but not limited to, a lithylene battery 4, which can be relatively flat shaped and preferably, relatively flexible. Other embodiments are provided with connecting means for the electricity network, for example a plug. A not shown battery holder is provided for holding at least one battery 4. For continuous use of the cover 1 it is advantageous if batteries 4 can be exchanged without interfering with the functioning of the cover 1, particularly the electroluminescent sources 3.

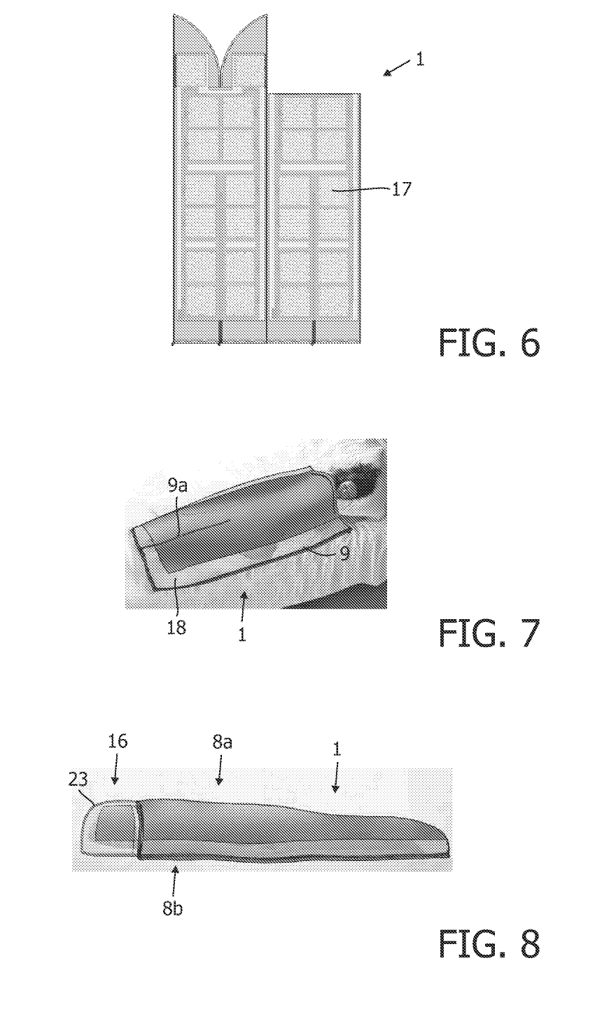

In the embodiment that is shown in FIG. 6, a detachable light permeable (translucent to the LED emitting wavelengths) lining 17 is attached on the inside of the cover 1, over the foils 2, so that possible heat that is generated by the LEDs 3 can be decreased. For example, the lining 17 may comprise phase change material. The lining 17 may add to the comfort of the cover 1. In an embodiment, the lining 17 comprises multiple layers that serve various purposes, also the lining 17 can be used instead of, or as a part of above mentioned sheet 8. For example, a liner 17 made of soft and bulky material may add to the comfort. In an embodiment, the lining 17 has fluid absorbing properties. In another embodiment, the lining 17 acts as a diffusing and/or light scattering layer for electroluminescent point sources 3. The lining 17 can for example be taken off the cover 1 for washing and/or exchanging. In different embodiments, the lining 17 comprises a fabric or textile, or any other polymer or rubbery substance in general. Embodiments of ‘non-woven’ fabric linings 17 comprise fibers that are bonded together, for example by use of resins or mechanical entanglement, as compared to woven fabric linings, where the fibers may be held together by weaving of the yarn and/or twisting of the fibers in the yarn. Suitable fibers include natural textile fibers, such as cotton or wool fibers, regenerated fibers, such as viscose, and synthetic fibers such as polyester, polyamide (nylon) or polyacrylic fibers. To get a more homogeneous light emitting surface, multiple layers may be provided, for example a layer of diffusing foam can be placed in between the flexfoils and the lining 17. In other embodiments, the lining 17 comprises non-woven or spacer fabrics or foam or any other material that diffuses light and create a homogenous light panel out of an assembly of point sources of light such as LEDs 3.

In other embodiments, the lining 17 is configured to withstand and/or absorb faeces and/or fluids coming from the user. For example, the lining 17 can include a diper or the like and/or has properties to repel and/or absorp moisture and bodily fluids. This lining 17 or a layer thereof can be disposable. This might for example be advantageous with an embodiment of a cover 1 for babies, providing a type of nappy function. The lining 17 can be exchanged and washed and the rest of the cover 1 can be kept relatively clean. In other embodiments, the cover 1 itself is configured to withstand, repel and/or absorp faeces and/or fluids and/or can be washed, such that the electric components, for example the electroluminescent sources 3 and/or the electric circuit, will not be damaged. In an alternative embodiment, the light panels can be encapsulated individually, collectively or in modules in a lining 17 material or for example in polymeric/elastomeric soft materials for protection and robustness. For this, protective layers may be provided in the cover 1, for example, rubbery or polymer materials. Of course multiple linings 17 can be provided for one cover 1.

The use of an embodiment of cover 1 can for example be as follows. A patient is placed on the cover 1 on the side of the LEDs 3, preferably on the sweat absorbing lining 17, that touches the naked skin. The LEDs 3 expose the body of the patient preferably with blue light. Preferably, the LEDs are configure to emit at a high-intensity narrow band blue light with a wavelength in the 450-470 nm spectrum, centered around a 450 nm peak and no light emission in the UV wavelength area. As shown in FIG. 2, the foil 2 contain holes, so that body heat and moisture can exit.

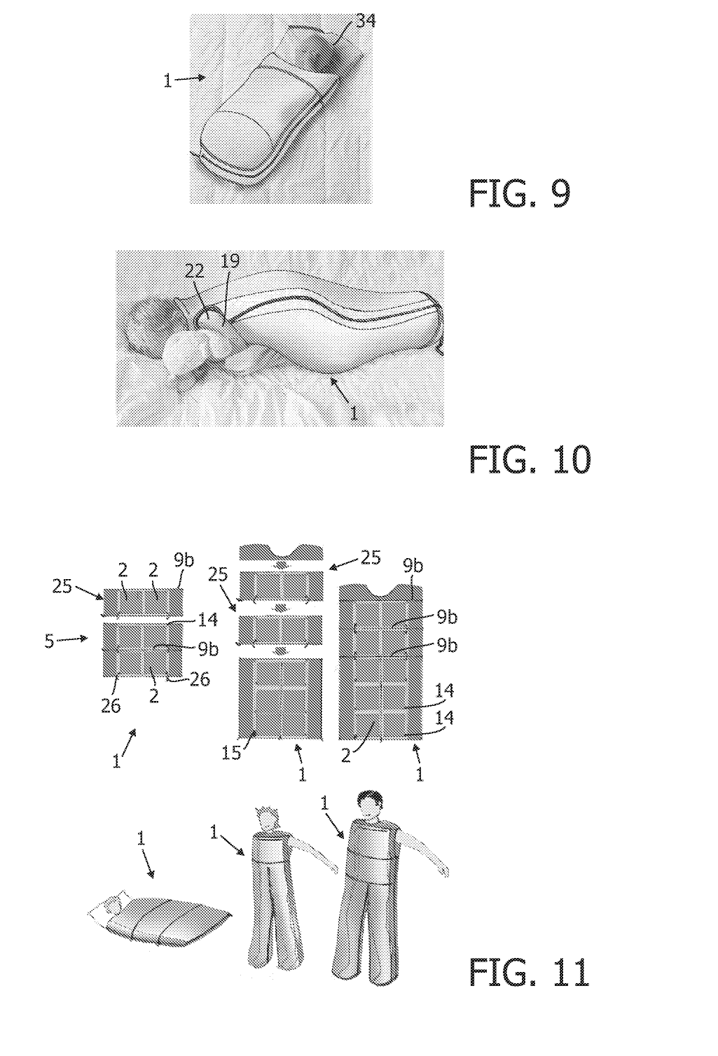

In an embodiment, when the patient is placed on the cover 1, the cover may be closed by means of joining means 9. Different embodiments of the joining means 9 comprise zippers 9, velcro, buttons and/or the cover 1 may be attached directly to the patient. When the joining means 9 are joined, a sleeping bag may be formed as shown in the embodiments of FIGS. 8 and 9. In an embodiment, the cover 1 is provided with a pillow 34, which may also comprise electroluminescent sources 3.

Different embodiments of the cover 1 have different sizes. An embodiment for babies is shaped for babies to one year old (FIG. 9), other embodiments are shaped as an infant's sleeping bag for 1 to 4 year old children and for example have removable cotton sleeves 19 (FIG. 10). In an embodiment a sleeping bag can be resized to three sizes for different treatments, different and/or growing patients, for example for the following age groups: 7 to 12 year old children, 12 to 18 year olds and adult users (FIG. 11).

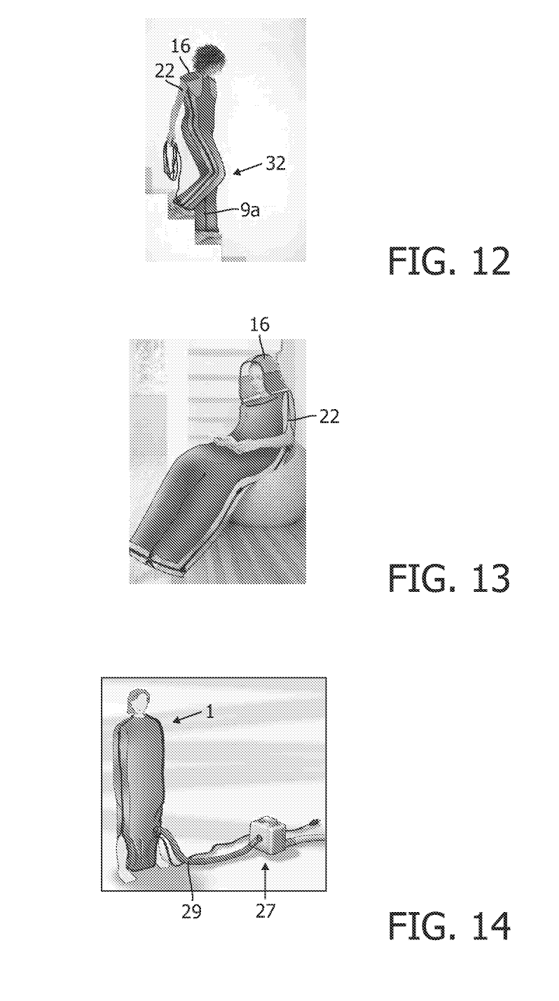

In an embodiment, the sleeping bags 1 can consist of two separate sheets 8a, 8b, each covered on one side with foils 2 (see FIG. 5). For example, zippers 9 are provided on the sides of the cover 1 and by zipping the sheets 8a, 8b a sleeping bag can be created. In particular embodiments, the upper part 20 of the sides have ‘pushbuttons’ 21 and by leaving these buttons 21 open, arm openings 22 are created (FIG. 12, 13). In the middle of both sheets 8 a zipper 9a is placed, to create pants 32, providing for a hybrid function for the cover 1. In an embodiment, no LEDs 3 are placed at knee and hip height, so that the user can comfortably move in the suit.

One of said sheets 8a is provided with flaps 16 containing two extra foils 2. In an embodiment, the flaps 16 are used as shoulder fasteners to expose the shoulders when the cover 1 is used as a suit, as shown in FIG. 12, and it can be used as a hood when it is used as a sleeping bag or suit, to enlighten the face, as shown in FIGS. 8 and 13. When used as a hood while sleeping, in a particular embodiment, two arcs 23 are provided, so that the hood doesn't touch the face, but is hanging on a distance above the head. This is shown in FIG. 8. The backing material of the hood is for example made of a net type material to create an open structure and air can flow through. The backing material could for example also comprise a porous material.

In an embodiment, at the bottom part of the cover 1 extra material 24 is attached that can be pulled up or down the leg. This may comprise elastic material to be able to clamp a part of the cover 1 against the leg.

In another embodiment the cover 1 is constructed by joining modular cover parts 25 with foils 2 to each other, as can be seen from FIG. 11. These building blocks are for example provided with two foils 2 placed side to side. In the shown embodiment, joining means 9 comprise zippers 9b on the edges with which the modular cover parts 25 can be connected to the cover or may build to form a cover 1. The LEDs may for example be connected by plugging small connectors 26. In another embodiment (not shown), the LEDSs 3 automatically connect to connectors 26 by joining the joining means 9b. Another embodiment is provided with conductive zippers 9b or Velcro, such that modular elements 25 connect to the electric circuit by joining the velcro. Other joining means 9b may of course also be conductive and applied for like embodiments. The embodiment provided with the modular parts 25 is for example advantageous when the user is a Crigler-Najjar patient. By means of the variable size, the patient can use the cover 1 for longer periods, for example several years and adjust the cover 1 for his comfort. The cover 1 can be enlarged by attaching a modular part 25 when the cover 1 becomes too small. In an embodiment of the cover 1, between the sheets 8a, 8b two extra modular cover parts, with or without foils 2, can be zipped to enlarge the size of the cover 1, to provide extra space for body movement during sleep.

In another embodiment the cover 1 can be of variable size by means of extra backing material, in which case the upper part of the cover 1 can be folded and, for example, hidden behind a zipper, for example hidden in a zipped pocket. When the user grows and the cover 1 becomes relatively too small, the extra material can be revealed and foils 2 can be placed inside. The extra material already contains the electronic connectors 26 and foil connectors 14 to connect the foil 2 to the system.

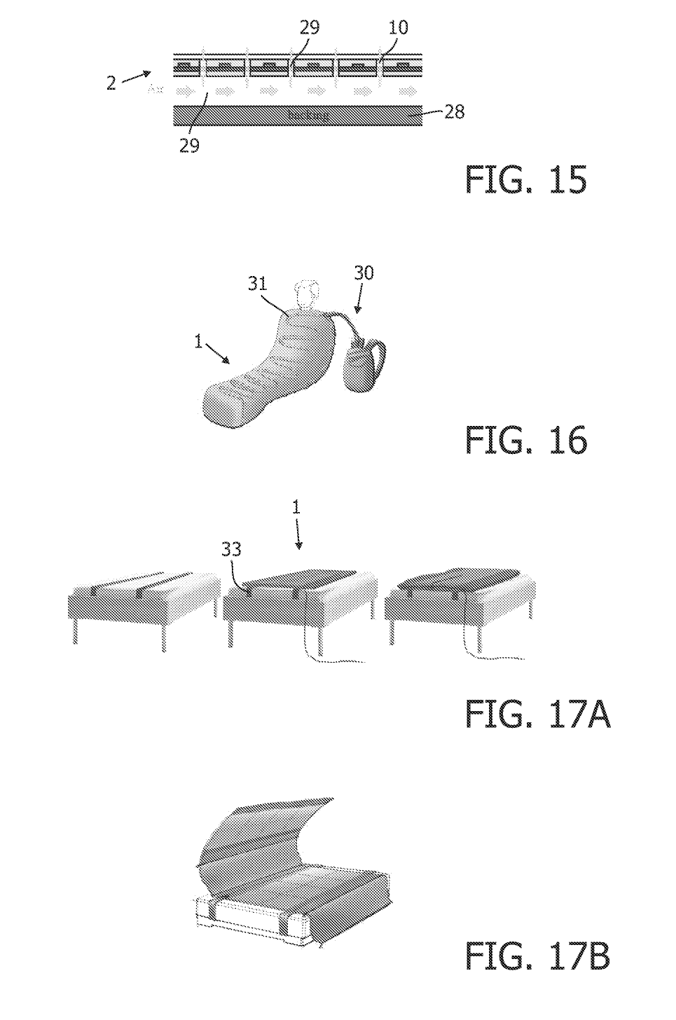

In an embodiment, the cover 1 is provided with a cooling system, for example when the cover 1 and/or the LEDs dissipate a relative large amount of heat. The cooling system for example comprises an airconditioning apparatus (airco) 27 that is connected to the suit and blows cold air 29 between a backing material 28 and the foils 2, as shown in FIGS. 14 and 15. In an embodiment, air also blows through the openings 10 in the foil 2 onto the body.

Another embodiment is provided with a liquid cooling system 30, for example as known from military suits. An example of such an embodiment is shown in FIG. 16. Cool water is pumped through tube frames 31 in the cover 1 to cool the electronics and/or body. To prevent the risk of overheating, an embodiment of the cover 1 comprises a built-in sensor that measures the temperature and for example decreases the light intensity when the temperature exceeds a certain level, until the desired temperature is reached again.

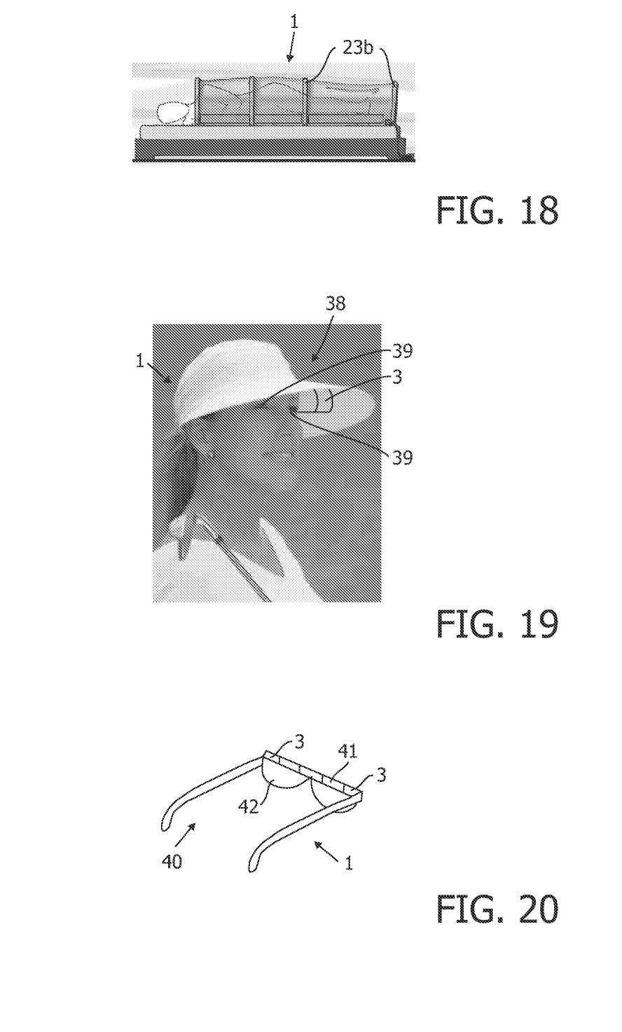

In another embodiment, cooling is provided passively by providing the cover 1 with a frame 23b of tubes, for example shaped as hoops or arcs, in the cover, so that an air layer is created between at least a part of the body and the cover 1, as shown in FIG. 18.

To prevent wrinkling and folding of the foils 2 because of the moving an embodiment of the cover 1 comprises fixation means, for example bands 33, that are for example fixed to one of the sheets 8 and can be placed for example around body support, such as a mattress. Such an embodiment is shown in FIGS. 17A and B. The bands 33 pull the sheet 8 tight. The user can now lie on the sheet 8 without the risk of folding it during sleep.

In an embodiment the cover 1, or at least the inside of the cover 1, is provided with reflecting material so that the light of the LEDs 3 is reflected and possibly more light reaches the part of the body. In an embodiment, light that is transmitted to the back of the LEDs 3, is reflected back to the front side so that it reaches the body. This will improve the light output and improve the efficiency of the cover 1.

In other embodiments the cover 1 comprises other electroluminescent sources 3 than the foil 2 with LEDs 3. For example OLEDs 3 and/or LEDs 3 attached to conductive and/or non-conductive yarns 11, 12 may be provided within the scope of the invention. Other LEDs 3 and/or other ways of attaching LEDs 3 and/or other electroluminescent sources to a substrate 2 are obviously possible within the scope of the invention.

In another embodiment the cover 1 is provided with at least one opening. Next to arm openings 22, in different embodiments openings are provided for the head, shoes, eyes, ears and/or mouth. In other different embodiments, openings are provided to pass through food, urine and/or faeces. In specific embodiments, the openings are reopenable and/or reclosable.

In yet other embodiments the control means 5 receive data from sensors. Sensors and/or other measurement devices are provided to provide data for the control means 5, which will send a signal to the adjustment, if necessary. For example, the status of the illness can be measured by measuring the colour of the skin. Also moisture conditions and/or temperature conditions can be measured with known devices. Another example is measuring the colour of the light, i.e. the wavelength, to check what wavelength is being emitted. In this way, the system, i.e. control means 5, can self-adjust in time, for example according to a pre-set schedule. Wavelength settings and/or intensity settings can be adjusted as well as the temperature and/or moisture conditions of the cover 1.

Blue light in a wavelength spectrum of approximately 440 to 480 nm, for example approximately 450 nm can be useful for revitalizing a person through the eyes. Therefore, in a different embodiment, a cover 1 comprises a hat, cap and/or visor 38, provided with electroluminescent sources 3, for example (O)LEDs 3. The blue light is emitted to the area 39 around the eyes, as can be seen from FIG. 19. Preferably, the blue light is emitted to the area around the bottom eyelids, from above the forehead. Therefore, an advantageous embodiment of a revitalising head cover 1 is provided with LEDs in the cap portion 39. Of course the light of the electroluminescent sources 3 can also be reflected to the area of the eyes 39. Another embodiment has the shape of glasses 40, where the LEDs can for example be provided in the portion 41 above the glasses 42, as can be seen from FIG. 20. Embodiments of the revitalising head covers 1 are provided with transparent electroluminescent sources 3, for example OLEDs 3, such that the electroluminescent sources are attached to a transparent surface, for example in the glasses 42 and/or a transparent visor. These types of revitalising head covers 1 can be revitalising, for example in tiring moments, for example when working at night, for example truck driving at night or other night shift work. There is little loss of comfort. The LEDs can be integrated with the fabric, for example such as explained above, for example by means of a foil 2 and/or integration of LEDs 3 with conductive and non-conductive yarns 11, 12. Flexible substrates and/or flexible electroluminescent sources 3 can be integrated with the fabric of the revitalising head cover 1. Of course OLEDs could be attached to the head cover as well. Different embodiments involve power supplies such as batteries, fuel cells, etc. Control means could be integrated as well. Attachment means for the electroluminescent sources for example include glue.

As mentioned above, phototherapy can be applied for wound healing. For example, wounds on women's breast, particularly around the nipples during breastfeeding, can also be treated by phototherapy. If these types of breast wounds are not treated in time, infections might occur and/or breastfeeding has to be interrupted. Since breastfeeding may take place for example every three or four hours and each session may take for example up to an hour, there might be insufficient time for the wound(s) to heal. Although for example for pain relief certain creams are sometimes used, those generally do not accelerate breast wound healing.

By applying an embodiment of a cover 1 for emitting light at certain wavelengths to women's breasts, the healing of the wounds, especially near the nipples, can be accelerated and/or wounds and/or infections may be prevented. Therefore it is advantageous when a cover 1 can be worn near the breasts, or at least near the nipples. Furthermore, embodiments of a cover 1 for breast wound healing may be used to prevent wounds or for cosmetic purposes.

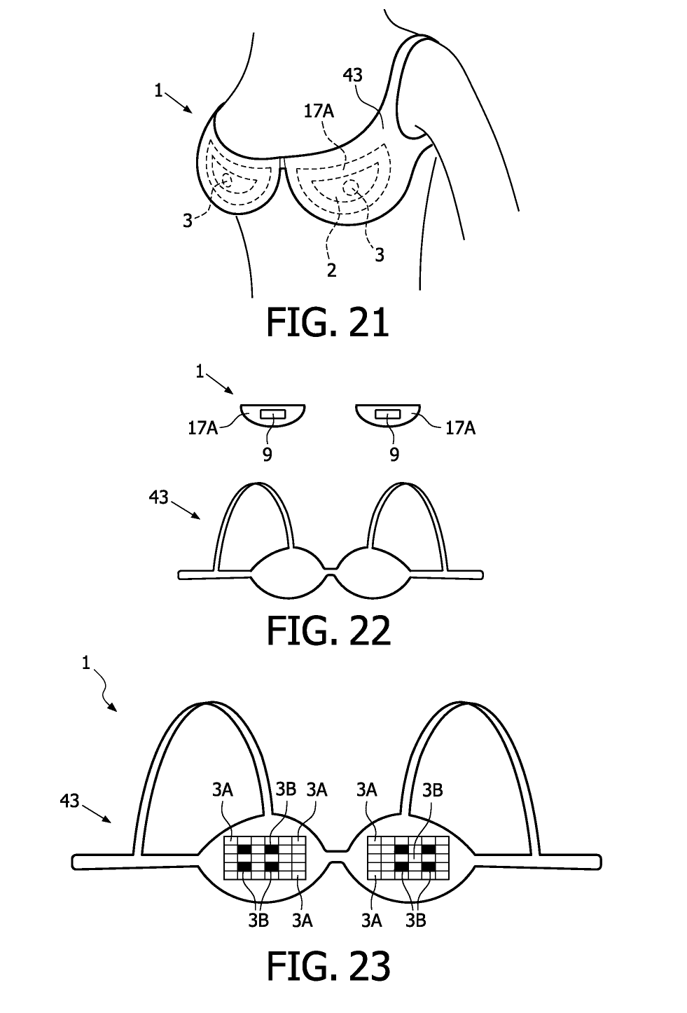

For example, an embodiment of a cover 1 is at least partly preshaped in the form of a nipple, possibly including at least part of the areola mammae and/or further breast. Also, the cover 1 for breast wound healing can be flexible, wherein it is configured to at least partly follow the curves of the female breast. In an advantageous embodiment, the cover 1 is constructed like a bra 43 or a replaceable lining 17A for a bra 43. In FIG. 21 a bra 43 is shown, which is provided with elements 2, 32, 17A that are indicated with dashed lines. This embodiment has advantageously combined electroluminescent sources like LEDs 3 with bras 43 so that a cover 1 functions as a phototherapeutic bra 43. The lining 17A may be applied, among others, for comfort, light scattering and for example absorption of leaking fluids from the breast. In an embodiment the electroluminescent sources comprise LEDs 3 that emit light in the red and/or infrared region, which is advantageous for wound healing. With the aid of control means 5, the wavelength and/or intensity of at least a part of the LEDs can be changed to apply a more optimised treatment. The bra 43 for example comprises foils 2, knitted, woven and/or non-woven textiles 2 and/or conductive and non-conductive yarns 11, 12 to which the LEDs 3 are attached, as already described above. The foil 2 with LEDs 3 can for example be attached to or laid in the inside of the bra 43, wherein for example a lining 17A or another type of layer acts as a diffusing layer. In another embodiment, the cover 1 for breast wound healing can be put in a compartment that is provided in a bra 43. In these and other ways, a phototherapeutic bra 43 can be conveniently worn like a conventional bra. Of course, the cover 1 doesn't need to be in direct contact with skin and one or more layers can be put in between the cover 1 and the skin.

In an embodiment, the lining 17A includes LEDs 3 and/or a light scattering and/or diffusing layer, and can be placed in the bra 43. The lining 17A may be fixedly integrated with the bra 43 or can be placed in a conventional bra 43 in a replaceable way, as shown in FIG. 22. For this, the lining 17A may comprise joining means 9, such as for example comprising Velcro-like material, buttons, etc. In another embodiment, the cover 1 comprises a nursing pad or another pad, to be put against a breast. A nursing pad according to this embodiment may prevent and/or absorb leakage and apply phototherapy by having LEDs 3 applied. In other embodiments, the cover 1 for women's breast is integrated or joinable with T-shirts, tops, patches, etc. To enhance the therapy, phototherapy can be applied in combination with creams and/or agents.

With the aid of sensors and control means 5, as mentioned above, the bra 43 can self adjusts its therapy according to the size and/or state of the wound(s) in time. For this, software and/or predetermined data are used so that the phototherapy can be performed according to measurements and predetermined data. For example, the light intensity of at least a part of the LEDs 3 can be adjusted. Sensors can be used that measure temperature, colour and/or emittance of the skin. In an embodiment, the LEDs 3 are configured to be light sources 3A as well as sensors 3B, as shown in FIG. 23. In an embodiment, the reflectivity of the skin is measured with the LEDs 3B that sense light that is emitted by the LEDs 3A and reflected by the skin. From this reflectivity information, the state of specific parts of the body, for example the nipples can be obtained by comparing with predetermined data and/or changes of the nipples in time, for example hours or days. In another embodiment, the lining 17A and/or LEDs 3 are shaped to fit around a nipple and/or the area around the nipple.

It shall be obvious that the invention is not limited in any manner to the exemplary embodiments that are represented in the description and the drawings. Many variation are possible within the framework of the invention as outlined by the claims. A cover 1 may for example configured for any type of phototherapy, not specifically described in the above text. Combinations of one or more aspects of the embodiments or combinations of the embodiments are possible within the framework of the invention. All comparable variations are understood to fall within the framework of the invention as outlined by the claims.

For instance, although in relation to wound healing a bra is discussed as one embodiment, other body covers discussed in the description can be used for wound healing purposes too.

Furthermore the body cover, according to the invention as used for wound healing can also be used for pain relief, e.g. for the treatment of muscle pain or joint pain.

1. Body cover, comprising a substrate, for covering at least part of a human body, and at least one electroluminescent source coupled to the substrate, for illuminating at least a part of the body, wherein the cover is configured to at least partly surround at least a part of the body and/or a body support.

2. Body cover according to claim 1, wherein the cover is substantially flexible and/or comprises at least one substantially flexible substrate.

3. Body cover according to claim 1, wherein the at least one electroluminescent source is configured to emit blue light between a wavelength of 440 nm and 480 nm, preferably between 445 nm and 470 nm, more preferably approximately 450 nm.

4. Body cover according to claim 1, wherein the at least one electroluminescent source comprises a LED (Light Emitting Diode) and/or OLED (Organic LED).

5. Body cover according to claim 1, comprising joining means for joining at least part of the cover to another part of the cover and/or part of a human body.

6. Body cover according to claim 1, wherein the joining means comprise closing means configured to have at least a part of the cover surround at least a part of a human body.

7. Body cover according to claim 1, comprising at least one detachable substrate and/or electroluminescent source.

8. Body cover according to claim 1, which is modular and/or wherein different parts are modular.

9. Body cover according to claim 1, wherein the cover comprises an element configured to cover at least a part of a head to illuminate at least one eye.

10. Body cover according to claim 1, wherein the cover comprises at least one opening for seeing through, breathing through, allowing at least one body part through and/or passing through specific elements, for example food.

11. Body cover according to claim 10, wherein the at least one opening comprises sealing means for substantially sealing said at least one opening around said at least one body part.

12. Body cover according to claim 1, comprising connecting means for connecting the at least one electroluminescent source to a power source.

13. Body cover according to claim 1, comprising at least one battery holder with connecting means, wherein the connecting means are configured to be connected to a power supply, while at least one battery is removed and/or replaced.

14. Body cover according to claim 1, having a thermal conditions and/or moisture conditions regulating configuration.

15. Body cover according to claim 1, comprising control means provided with wavelength settings and/or intensity settings adjusting means, for the at least one electroluminescent source, said means preferably comprising local settings adjustment means.

16. Body cover according to claim 14, wherein control means are provided, comprising at least a processing circuit, wherein the processing circuit is configured to adjust and/or monitor at least one of said conditions and/or settings, preferably by measuring said at least one of said settings and/or conditions, comparing at least one measured value to at least one pre-set value and adjusting/keeping said at least one of said settings and/or conditions in correspondence with the pre-set value.

17. Body cover according to claim 8, wherein said modular cover(s) and/or modular cover part(s) are configured to connect to the connecting means and/or control means.

18. Body cover according to claim 17, wherein the joining means are configured to connect said modular cover(s) and/or modular cover parts(s) by joining the joining means.

19. Body cover according to claim 1, wherein openings are provided in the at least one substrate and/or in the at least one electroluminescent source and/or between multiple electroluminescent sources.

20. Body cover according to claim 1, wherein the at least one substrate comprises at least one sheet of foil, polymer, particularly polymeric textile material, knitted woven and/or non-woven textile, which may be a polymer, diffusive and/or scattering materials and/or spacer fabrics, wherein the at least one electroluminescent source is attached to the at least one sheet and/or between the at least one sheet.

21. Body cover according to claim 20, wherein said sheet comprises at least one partly conductive yarn, wherein the at least one electroluminescent source is attached to and/or between at least partly conductive yarns.

22. Body cover according to claim 1, wherein the cover comprises one or more detachable layers.

23. Body cover according to claim 1, wherein the at least one substrate and/or the detachable layer is configured to withstand, repel and/or absorb fluids and/or faeces.

24. Body cover according to claim 1, wherein the cover comprises a frame for keeping the cover in a substantially pre-shaped condition and/or for allowing temperature regulating means to pass through.

25. Body cover according to claim 1, wherein fixation means are provided to be able to keep at least a part of the cover in place during use.

26. Modular element of a cover according to claim 1 which is configured to be joined to like modular elements by means of joining means.

27. Glasses and/or at least partial head cover, especially forming a cover according to claim 1, connected and/or provided with a power supply for supplying power to the at least one electroluminescent source, wherein said glasses and/or said at least partial head cover are configured to have the at least one electroluminescent source emit light approximately into the eyes, preferably approximately near the bottom eye lids, wherein the at least one electroluminescent source has a wavelength between 440 nm and 480 nm, preferably between 447 nm and 470 nm, more preferably approximately 450 nm.

28. Method for revitalising persons, comprising carrying glasses and/or a partial head cover according to claim 27 on the head and emitting blue light from said glasses and/or partial head cover into the eyes, having a wavelength between 440 nm and 480 nm, preferably between 447 nm and 470 nm, more preferably approximately 450 nm.

29. Method for radiating at least part of a human body, especially in the treatment of jaundice and/or crigler najjar, comprising covering at least part of the body and/or a body support with a cover with at least one electroluminescent source and emitting light from the electroluminescent source to at least part of the body.

30. Method according to claim 29, comprising surrounding at least part of the body by the cover.

31. Method according to claim 29, comprising joining cover ends.

32. Method according to claim 29, comprising joining modular cover ends.

33. Method according to claim 29, comprising measuring at least one of body colour, temperature, humidity, wavelength and/or intensity of the light.

34. Method according to claim 33, comprising comparing at least one of the measured values with at least one pre-set value and adjusting the wavelength and/or intensity of the at least one electroluminescent source according to the pre-set.

35. Method according to claim 34, comprising adjusting the wavelength of at least one electroluminescent source locally, whereas another (part of) the electroluminescent source emits at a substantially different wavelength then the adjusted wavelength value, or doesn't emit. Method for healing and/or suppressing jaundice and/or crigler najjar, comprising covering at least part of the body and/or a body support with a cover with at least one electroluminescent source and emitting light from the electroluminescent source to at least part of the body.

36. Use of a body cover, comprising a substrate, for covering at least part of a human body, and at least one electroluminescent source coupled to the substrate, for illuminating at least a part of the body, wherein the cover is configured to at least partly surround at least a part of the body and/or body support, for the treatment of blood related diseases, especially jaundice and crigler najjar.

37. Body cover according to claim 1, wherein the cover is a breast cover for covering at least part of a women's breast, and wherein it is at least partially configured to follow the curves of women's breasts, preferably to function as a bra.

38. Body cover according to claim 37, comprising a lining that fits in and/or is joinable with a bra.

39. Body cover according to claim 37, comprising a light diffusing layer.
